(19) United States
(12) Patent Application Publication
(10) Pub. No.: US 2004/0258249 A1
(43) Pub. Date: Dec. 23, 2004

(54) Method for operating a hearing aid device and hearing aid device with a microphone system in which different directional characteristics can be set

(76) Inventors: Torsten Niederdrank, Erlangen (DE); Tom Weidner, Erlangen (DE)

Correspondence Address: SCHIFF HARDIN, LLP, PATENT DEPARTMENT, 6600 SEARS TOWER, CHICAGO, IL, 60606-6473, US

(21) Appl. No.: 10/871,712

(22) Filed: Jun. 18, 2004

(30) Foreign Application Priority Data
Jun. 20, 2003 (DE) 103 27 891.5

(51) Int. Cl. H04R 29/00; A61B 5/00

(52) U.S. Cl. 381/60; 600/559

(57) ABSTRACT

The sound quality of a hearing aid device with a directional microphone system is to be improved. For this purpose, the microphone signals of microphone units with directivities of different orders are analyzed and divided into frequency bands. A weighting of the microphone signals that are emitted from the microphone units with directivities of different orders takes place in the individual frequency bands. The weighting takes place in particular in dependence on the signal level of the microphone signals.

### BACKGROUND OF THE INVENTION

The invention relates to a method for operating a hearing aid device with a microphone system, a signal processing unit and an output transducer, the microphone system comprising at least two microphone units from which microphone signals are emitted and which have directional characteristics of different orders. Furthermore, the invention relates to a hearing aid device for carrying out the method.

In modern hearing aid devices, devices for the classification of hearing situations are used. Depending on the hearing situation, the transmission parameters of the hearing aid device are varied automatically. Among the things that may be influenced by the classification are the operating mode of interfering noise suppression algorithms and the microphone system. For example, according to the hearing situation that is detected, a selection is made (discretely switched over or continuously cross-faded) between an omnidirectional directional characteristic (directional characteristic of the zeroth order) and a definite directivity of the microphone system (directional characteristic of the first or higher order).

To produce the directional characteristic, gradient microphones are used or a number of omnidirectional microphones are electrically interconnected. Microphone systems of this type have a frequency-dependent transmission mode, in which a distinct drop toward low frequencies can be noted. By contrast, the noise response of the microphones is frequency-independent and slightly increased in comparison with an omnidirectional microphone. To achieve a natural sound impression, the high-pass frequency response of the microphone system must be balanced by boosting the low frequencies. In the process, the noise present in the low frequency range is likewise boosted at the same time and under some circumstances becomes distinctly and disturbingly audible, whereas quiet sounds are concealed by the noise.

International Patent Publication No. WO 00/76268 A2 discloses a hearing aid device with a signal processing unit and at least two microphones which can be interconnected to form directional microphone systems of different orders, it being possible in turn for the directional microphone systems to be interconnected with a weighting dependent on the frequency of the microphone signals emitted by the microphones. Depending on the result of a signal analysis, the cut-off frequency between neighboring frequency bands for which a different weighting of the microphone signals is provided can be set.

German Patent Document No. DE 197 03 228 A1 discloses a method for boosting input signals of a hearing device in which a compression of the signals picked up by the hearing device is performed using an AGC (Automatic Gain Control) circuit depending on the recordable signal level. In addition to the recording of the signal level of the input signal, a signal analysis is carried out for the detection of the acoustic situation and, on the basis of the result of the signal analysis, the mode of the compression is adaptively varied. The signal analysis and the compression may also be carried out in parallel in different frequency bands.

European Patent Document No. EP 0 942 627 A2 discloses a hearing device with a directional microphone system having a signal processing device, an earphone, and a number of microphones, the output signals of which can be interconnected with different weightings to generate an individual directional microphone characteristic by way of delay devices and the signal processing device. In the case of the directional microphone system, the preferred receiving direction (main direction) can be individually set adapted to a prevailing hearing situation.

U.S. Pat. No. 5,524,056 discloses a hearing device with an omnidirectional microphone and a directional microphone of the first or higher order. The microphone signal of the directional microphone is boosted in its amplitude in the range of low signal frequencies and equalized with the microphone signal of the omnidirectional microphone. Both the microphone signal of the omnidirectional microphone and the microphone signal of the directional microphone are fed to a switching unit. In a first switching position of the switching unit, the omnidirectional microphone is connected to a hearing device amplifier and, in a second switching position of the switching unit, the directional microphone is connected to said hearing device amplifier. The switching unit can switch over automatically in dependence on the signal level of a microphone signal.

A disadvantage of the known hearing aid devices with a directional microphone system is that, in certain hearing situations, either the directivity of the microphone system is not optimally used or a high degree of directivity leads to distinctly audible deterioration in the sound quality.

### SUMMARY OF THE INVENTION

The object of the present invention is to provide a better sound quality of a hearing aid device with a directional microphone system.

In the case of a method for operating a hearing aid device with a microphone system, a signal processing unit and an output transducer, the microphone system comprising at least two microphone units from which microphone signals are emitted and which have directional characteristics of different orders, this object is achieved by the following method steps: carrying out a signal analysis in the case of at least one microphone signal for determining signal properties at specific frequencies or within specific frequency bands, utilizing a different weighting of the microphone signals that are emitted with different directional characteristics from the microphone units, depending on the result of the signal analysis and the frequency of the microphone signals.

Furthermore, in the case of a hearing aid device for carrying out the method with a microphone system, a signal processing unit and an output transducer, the microphone system comprising at least two microphone units from which microphone signals are emitted and which have directional characteristics of different orders, the object is achieved by a mechanism for splitting the microphone signals of the microphone units with directional characteristics of different orders into a number of frequency bands, a signal analyzer in the case of at least one of the microphone signals and also a mechanism for the different weighting of the microphone signals in the individual frequency bands depending on the result of the signal analysis.

The hearing aid device according to the invention comprises a microphone system with at least two microphones to be able to realize directional characteristics of the zeroth and first order. However, more than two microphones are preferably present so that directional characteristics of the second and higher orders are also possible. Furthermore, the hearing aid device comprises a signal processing unit for the processing and frequency-dependent boosting of the microphone signal generated by the microphone system. The signal output usually takes place by an acoustic output signal via an earphone. However, other output transducers, for example those producing vibrations, are also known.

A directional characteristic of the zeroth order is to be understood as meaning an omnidirectional directional characteristic which is produced, for example, by a single omnidirectional microphone that is not connected to further microphones. A microphone unit with a directional characteristic of the first order (“directional microphone of the first order”) may be realized, for example, by a single gradient microphone or the electrical connection of two omnidirectional microphones. With directional microphones of the first order, a theoretically achievable maximum value of the directivity index (DI) of 6 dB (hypercardioid) can be achieved.

In practice, DI values of 4-4.5 dB are obtained on the KEMAR (a standard research dummy) with optimum positioning of the microphones and the best equalization of the signals generated by the microphones. Directional microphones of the second and higher orders have DI values of 10 dB and more, which are advantageous, for example, for better speech intelligibility. If a hearing aid device includes a microphone system with, for example, three omnidirectional microphones, microphone units with directional characteristics of the zeroth to second order can be realized simultaneously on this basis by a suitable connection of the microphones.

A single omnidirectional microphone itself represents a microphone unit of the zeroth order. If, in the case of two omnidirectional microphones, the microphone signal of one microphone is delayed, inverted and added to the microphone signal of the other microphone, a microphone unit of the first order is produced. If, in turn, in the case of two microphone units of the first order, the microphone signal of one microphone unit is delayed, inverted and added to the microphone signal of the second microphone unit of the first order, a microphone unit with a directional characteristic of the second order is obtained. In this way, microphone units of any desired order can be realized—dependent on the number of omnidirectional microphones.

If a microphone system comprises microphone units of different orders, it is possible to switch between different directional characteristics, for example, by switching one or more microphones on or off. Furthermore, any desired mixed forms between the directional characteristics of different orders can be produced by suitable electrical connection of the microphone units. For this purpose, the microphone signals of the microphone units are weighted differently and added before they are further processed in the signal processing unit of the hearing aid device and boosted. In this way, a continuous, smooth transition can be realized between different directional characteristics, whereby disturbing artifacts during switching over can be avoided.

In the case of the hearing aid device according to embodiments of the invention, a signal analysis in which specific properties of the microphone signal are established is advantageously performed in the case of at least one microphone signal. What is important in the case of the signal analysis in connection with these embodiments is that these signal properties are determined in dependence on the signal frequency. As a result, it is possible to adjust the weighting of microphone signals that are emitted from microphone units with different directivities adaptively to the respective hearing situation depending on the result of the signal analysis.

So, in every frequency range a directivity can be set that is optimized for the respective frequency range. As a result, as much directivity as possible can be allowed, without the proportion of noise in the output signal of the hearing aid device that is generated by the microphone system being perceived as disturbing. This effect produced is achieved by a directivity being produced only in the frequency ranges of the useful signal in which increased microphone noise in any case causes a slight deterioration in the sound for the wearer of the hearing device. If, for example, in the “conversation” hearing situation, a high signal level is established merely in the frequency range between 1 kHz and 3 kHz, in this frequency range the microphone signal from the microphone unit with the highest order is given the greatest weight. In the other frequency ranges with lower signal levels, a directivity is advantageously foregone, at least to a great extent, by way of a corresponding weighting of the microphone signals.

In the signal analysis, the signal level of the microphone signal is preferably determined in dependence on the signal frequency.

The following settings of the microphone system can then be broadly derived from this: in the case of a high signal level of the microphone signal, the microphone noise is covered by the input signal and is not perceived as disturbing. So, in such a hearing situation, a high order of the directivity that is achievable with the microphone system can be set. By contrast, with a very quiet input signal, the situation is different. Here, the microphone noise induced by the directivity of the microphone system may be perceived as disturbing. It is therefore expedient in such a hearing situation to forgo the directivity, at least to a great extent, and merely further process the omnidirectional microphone signal or reduce the weight of the microphone signals from microphone units of a higher order.

For recording the signal level of the acoustic input signal in the case of a hearing aid device according to an embodiment of the invention, the microphone system is advantageously directly assigned a measuring and control unit. Apart from the direct level measurement, other measurements which are in direct relationship with the signal level of the input signal and allow conclusions with respect to the latter to be drawn can also be carried out: for example, measurement of the root mean square RMS. On the basis of the value measured in this way, the measuring and control unit controls the directional characteristic of the microphone system.

Advantageously, the directivity of the microphone system is automatically reduced in the case of low signal levels of the acoustic input signal. In particular, in the case of low input signal levels, an omnidirectional directional characteristic of the microphone system is set. In this way, troublesome microphone noise, which is perceived as disturbing, particularly in the case of low signal levels, can be prevented.

Apart from the signal level, however, a series of further signal properties can also be measured, for example, the modulation frequency or the modulation depth. Further examples are the slope of the envelope or the characteristic of the zero crossing. According to embodiments of the invention, the determination of these signal properties takes place in dependence on the signal frequency. The signal to be analyzed for this is advantageously divided into a number of frequency bands. Then, the directivity which, according to the result of the signal analysis, is of particular significance for the wearer of the hearing device, is increased in the frequency range or in the frequency ranges of the input signal. This may be, for example, a frequency range in the case of which the result of the modulation analysis indicates a voice signal. The weighting of the microphone signals may also be based on a combinational evaluation of a number of signal properties, for example, the signal level and the modulation frequency.

In the case of one embodiment of the invention, the microphone signal generated by the omnidirectional microphone unit is analyzed. This has the advantage that sound signals entering the microphone system from different directions are equally taken into account in the signal analysis. In the case of another embodiment of the invention, the microphone signal of one directional microphone is analyzed. This may be advantageous, for example, in the “conversation” hearing situation, in which the person conversing with the wearer of the hearing aid is assumed to be in the viewing direction and therefore the microphone signal of a microphone unit pointed in this direction is advantageously analyzed. However, the best results in the analysis of the sound field at a given instant are obtained when the microphone signals of a number of microphone units are evaluated simultaneously.

In the case of modern hearing aid devices, the microphone signal to be processed is usually first divided into frequency bands. In connection with the invention, in the case of one embodiment, the output signals of the individual microphone are first divided into individual frequency bands. Subsequently, the microphone signals in the individual frequency bands are interconnected to produce microphone units with directional characteristics of different orders.

Another embodiment of the invention provides that microphone units which differ with regard to their directional characteristics are first provided, in order subsequently to divide the output signals of these microphone units into frequency bands. The frequency-dependent different weighting of the microphone signals of the microphone units of different orders then also advantageously takes place in these frequency bands, it preferably being possible for both the weights of the microphone signals of different microphone units in one frequency band and the weights of the microphone signals emitted by one microphone unit in different frequency bands to be set independently of one another.

Furthermore, the analysis of the microphone signal or signals preferably takes place in parallel in the individual frequency bands. This is advantageous particularly because the achievement of a directivity in the low frequency range is in any case problematical. The microphone system can be set in such a way that, in the low-frequency frequency bands, it only acts as a directional microphone at very high signal levels and is merely set for omnidirectional reception at lower signal levels. In frequency bands with higher frequencies, on the other hand, a directivity of the microphone system may already be activated at lower signal levels.

The possibility of being able to weight and add the microphone signals emitted by microphone units of different orders in virtually any way desired allows any desired intermediate stage between the individual orders also to be set. As a result, abrupt switching over between different orders and the associated switching artifacts can be avoided. In particular, when changing the hearing situation, the weights of the individual microphone signals are also advantageously not changed abruptly from an initial value to a new end value, but are equalized very gradually.

The invention can be used in the case of all known types of hearing aid devices with a directional microphone system, for example, in the case of hearing aid devices which can be worn behind the ear, hearing aid devices which can be worn in the ear, implantable hearing aid devices or pocket hearing devices. Furthermore, the hearing aid device according to the invention may also be part of a hearing device system comprising a number of devices for assisting a person who is hard of hearing, for example, part of a hearing device system with two hearing aid devices worn on the head for binaural assistance or part of a hearing device system comprising a device which can be worn on the head and a processor unit which can be worn on the body.

### DESCRIPTION OF THE DRAWINGS

Further details and advantages of the invention emerge from the description which follows of exemplary embodiments.

FIG. 1 is a block diagram of a hearing aid device with a microphone system according to the invention,

FIG. 2 is a graph illustrating the directivity of the microphone system in dependence on the input signal level.

### DETAILED DESCRIPTION OF THE PREFERRED EMBODIMENTS

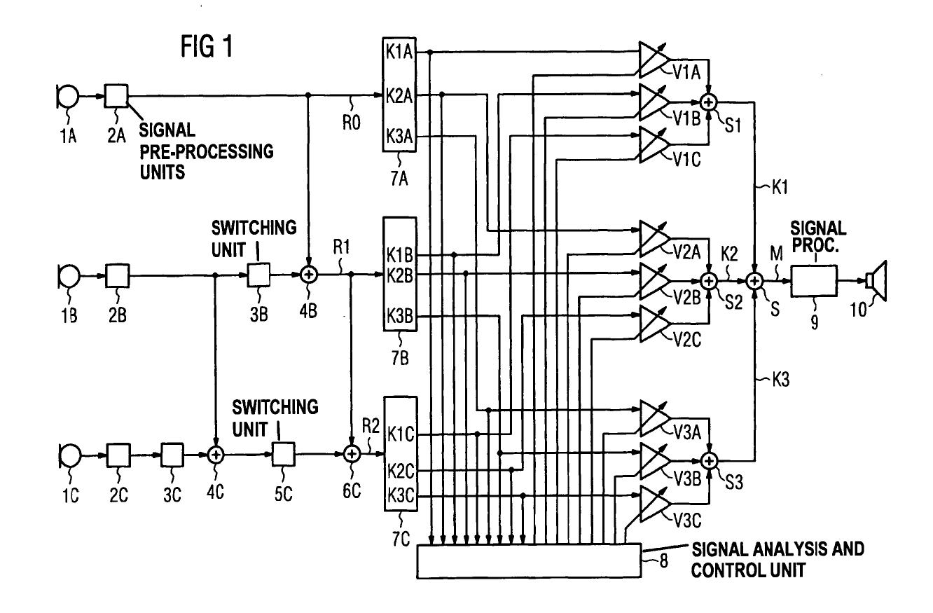

FIG. 1 shows a simplified block diagram of a hearing aid device with a directional microphone system. The directional microphone system comprises three omnidirectional microphones 1A, 1B, and 1C. Connected directly downstream of the omnidirectional microphones 1A, 1B, and 1C there is respectively a signal preprocessing unit 2A, 2B and 2C, which performs, for example, an A/D conversion and a signal preamplification.

The two microphones 1A and 1B are electrically connected to form a microphone unit with a directional characteristic of the first order. For this purpose, the microphone signal emitted by the omnidirectional microphone 1B is delayed and inverted in a switching unit 3B and, in a way similar to the microphone signal emitted by the microphone 1A, fed to an adder 4B. This turns the two microphones 1A and 1B into a microphone unit with a directional characteristic of the first order, from which the microphone signal R₁ is produced.

Similarly, by delay and inversion of the microphone signal emitted by the microphone 1C and addition of the microphone signal emitted by the microphone 1B in the adder 4C, the two omnidirectional microphones 1B and 1C also form a microphone unit with a directional characteristic of the first order. If, in turn, the microphone signal emitted by the adder 4C is delayed and inverted in the switching unit 5C and added to the microphone signal R1, a microphone unit with a directional characteristic of the second order is formed as a result. This produces the microphone signal R2 at the output of the adder 6C. The output signal of the omnidirectional microphone 1A with the directional characteristic of the zeroth order is designated by R0.

For dividing the microphone signals R0, R1 and R2 into frequency bands, the microphone signal R0 is fed to a filter bank 7A, the microphone signal R1 is fed to a filter bank 7B and the microphone signal R2 is fed to a filter bank 7C. In the exemplary embodiment, the three filter banks 7A, 7B and 7C lead to a splitting of the respective microphone signal into three frequency bands that are adjacent to one another. In the process, a division of the respective microphone signal into the same frequency bands takes place in each filter bank.

At the output of the filter bank 7A are the microphone signals K1A, K2A and K3A. By analogy, the microphone signals at the output of the filter bank 7B are designated by K1B, K2B, K3B and the microphone signals at the output of the filter bank 7C are designated by K1C, K2C and K3C. The output signals of the filter banks 7A, 7B and 7C are fed to a signal analysis and control unit 8 for evaluation. In this unit, the microphone signals of the microphone units with directional characteristics of different orders in the different frequency bands are analyzed. The signal analysis comprises, in particular, the determination of the signal level of the respective microphone signals. However, other characteristic signal variables, such as the modulation frequency, the modulation depth, the slope of the envelope or the characteristic of the zero crossing, may also be determined and evaluated.

The result of the signal analysis is used in the signal analysis and control unit 8 to calculate control parameters via which the directional characteristic in the individual frequency bands can be set. For this purpose, the output signals of the filter banks 7A, 7B and 7C are respectively fed to an amplifier V1A, V1B, V1C and V2A, V2B, V2C and V3A, V3B, V3C. The respective amplification by the amplifiers is set by the signal analysis and control unit 8 via the parameters calculated. As a result, the directional characteristic in the individual frequency bands is optimized.

Preferably, the directional characteristic is thereby set in such a way that there is a directivity that is as high as possible, without, however, causing an increase in the microphone noise that is perceived to be disturbing. Following the different weighting of the microphone signals of the directional microphones of different orders in the individual frequency bands by the amplifiers V1A to V3C with amplification that can be set, the output signals of the amplifiers are first added within the frequency bands with adders S1, S2 and S3, whereby the three microphone signals K1, K2 and K3 are produced. These in turn are fed to an adder S, at the output of which the microphone signal of the microphone system can be picked off. This is fed by the hearing aid device to a signal processing unit 9 for further processing and amplification. For conversion into an acoustic signal, the resultant output signal is finally fed to an earphone 10, from which the acoustic output signal is emitted into the auditory canal of a person wearing the hearing device.

The possibility of being able to weight the microphone signals of the directional microphones of the zeroth to second orders differently allows any desired order between the zeroth and the second order to be set in the case of a hearing aid device with the microphone system shown, that is to say, even any desired “intermediate order” (i.e., non-integer multiple). The degree of directivity can consequently vary as desired between the highest order and no directivity, with all intermediate stages also being included. So, the optimum degree of directivity can be set for each input signal with the microphone system concerned. In this case, it must be taken into account that the optimum degree of directivity may also be dependent on the individual hearing loss of a person wearing a hearing device. The individual profile of the characteristic directivity curves is obtained particularly by taking characteristic audiological variables into account, such as the hearing threshold in quiet conditions of a person wearing a hearing device in the individual frequency ranges, or taking hearing device settings into account, such as an automatic gain control AGC, customary in the case of hearing aid devices, or the cross section of a ventilation opening.

The microphone system makes it possible to set an individual profile of the directivity in dependence on the input signal. Hard “switching” between different directivities and the associated switching artifacts when there is a change in the hearing situation are avoided as a result.

In the case of the exemplary embodiment according to FIG. 1, the microphone signals amplified in the amplifiers V1A to V3C are added and fed to a signal processing unit 9 for further processing. In the signal processing unit 9, the frequency-dependent amplification of the microphone signal M takes place to compensate for the individual hearing loss of the person wearing the hearing device. This signal processing also preferably takes place in different frequency bands (channels) of the signal processing unit 9. In this case, these frequency bands may also be advantageously independent of the division produced by the filter banks 7A to 7C with regard to number and channel limits. Alternatively, however, the different amplification of individual frequency bands to compensate for the hearing loss may likewise already be undertaken by the amplifiers V1A to V3C. The signal analysis and control unit 8 is to be programmed correspondingly for this purpose. In this case, merely a signal post-processing, for example, final amplification and D/A conversion, takes place in the signal processing unit 9.

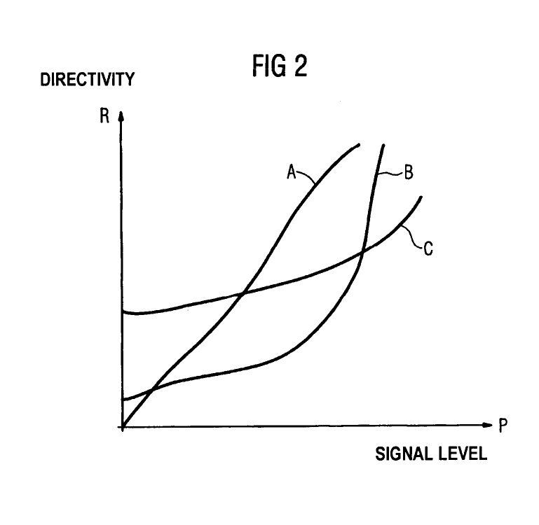

FIG. 2 illustrates examples of different directivities R in a frequency band in dependence on the signal level P in this frequency band. The directivity R may in this case assume any desired values between an omnidirectional (non existent) directivity and the maximum directivity that can be achieved with the microphone system. In the case of the characteristic curve A, there is no directivity in the case of a very low signal level in the frequency band concerned. However, the directivity increases virtually linearly as the level in the frequency band increases until, as from a certain level, the maximum directivity is achieved.

As a difference from the characteristic curve A, the characteristic curve B initially shows only a slight increase in the directivity as the signal level in the respective frequency band increases. Only in the case of very high signal levels is there a very steep increase in the directivity. Such a nonlinear characteristic curve should be set particularly in a low-frequency frequency band, since directional microphones act as low-pass filters and therefore a quiet low-frequency input signal requires great amplification, which leads to increased noise. Therefore, in the low-frequency range, the directivity is advantageously used only when there is a high signal level of the input signal in this frequency range and therefore only a slight amplification is required.

In the case of a directivity according to the third characteristic curve C, a relatively high directivity is already set in the case of a low signal level. In return, the increase in the directivity as the input level increases is less than in the two previous examples. Such a directivity in dependence on the signal level is advantageous particularly for a higher frequency range, since, as a result of the high-pass characteristic of the directional microphone system, in this range only a slight increase in the microphone noise is caused by the high directivity, even in the case of a low signal level of the input signal and a high amplification by the hearing aid device.

Apart from the signal level, other signal properties can also influence the profile of the directivity, such as the modulation frequency. In particular, the directivity set at a given instant may also be dependent on a number of parameters at the same time.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, e.g., memory elements, processing elements, logic elements, look-up tables, and the like, which may carry out a variety of functions under the control of one or more microprocessors or other control devices. Similarly, where the elements of the present invention are implemented using software programming or software elements the invention may be implemented with any programming or scripting language such as C, C++, Java, assembler, or the like, with the various algorithms being implemented with any combination of data structures, objects, processes, routines or other programming elements. Furthermore, the present invention could employ any number of conventional techniques for electronics configuration, signal processing and/or control, data processing and the like.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. A method for operating a hearing aid device with a microphone system, a signal processing unit and an output transducer, the microphone system comprising at least two microphone units from which microphone signals are emitted and which have directional characteristics of different orders, the method comprising:
carrying out a signal analysis of at least one microphone signal for determining signal properties at specific frequencies or within specific frequency bands, thereby producing a signal analysis result; and
applying a different weighting of the microphone signals that are emitted with different directional characteristics from the microphone units depending on the result of the signal analysis and a frequency of the microphone signals.

2. The method as claimed in claim 1, further comprising:
dividing the microphone signals that are emitted by the microphone units into a number of frequency bands and the different weighting of the microphone signals taking place in individual frequency bands.

3. The method as claimed in claim 1, further comprising analyzing the microphone signals that are emitted by the microphone units in the individual frequency bands.

4. The method as claimed in claim 1, wherein the analysis of the at least one microphone signal comprises determining at least one of a modulation frequency, a modulation depth, a slope of an envelope, and a characteristic of the zero crossing.

5. The method as claimed in claim 1, wherein the analysis of the at least one microphone signal comprises determining a signal level.

6. The method as claimed in claim 5, further comprising increasing the weight of a microphone signal of a microphone unit with a directional characteristic of a higher order in comparison with the weight of a microphone unit with a directional characteristic of a lower order as the signal level increases.

7. The method as claimed in claim 1, wherein the applying a different weighting comprises utilizing a hearing threshold in quiet conditions of a person wearing a hearing device who is assisted by the hearing aid device.

8. The method as claimed in claim 1, wherein the applying a different weighting comprises utilizing hearing device settings, including a cross section of a ventilation opening.

9. A hearing aid device, comprising:
a microphone system comprising at least two microphone units from which microphone signals are emitted and which have directional characteristics of different orders;
a microphone signal splitter configured for splitting the microphone signals of the microphone units with directional characteristics of different orders into a number of frequency bands;
a signal analyzer configured for carrying out a signal analysis in a case of at least one of the microphone signals;
a weighting mechanism configured for a different weighting of the microphone signals in the individual frequency bands in dependence on the result of the signal analysis;
a signal processing unit configured to process and create a digital output signal; and
and an output transducer for converting the digital output signal to a n audio output signal.
